(19) United States
(12) Patent Application Publication
(10) Pub. No.: US 2008/0249034 A1
(43) Pub. Date: Oct. 9, 2008

(54) Use of Macrolides for Treating Intestinal Inflammation

(75) Inventors: Mario Del Tacca, Pisa (IT); Corrado Blandizzi, Pisa (IT); Gabriele Morazzoni, Lainate(MI) (IT)

Correspondence Address: LUCAS & MERCANTI, LLP, 475 PARK AVENUE SOUTH, 15TH FLOOR, NEW YORK, NY, 10016, US

(73) Assignee: ZAMBON S.P.A., Bresso (IT)

(21) Appl. No.: 11/908,550

(22) PCT Filed: Mar. 14, 2005
(86) PCT No.: PCT/EP06/60709

§ 371 Date: May 15, 2008

(30) Foreign Application Priority Data
Mar. 21, 2005 (EP) 05102244.0
Mar. 14, 2006 (EP) PCT/EP2006/060709

(51) Int. Cl. A61K 31/7048; A61P 1/00

(52) U.S. Cl. 514/29

(57) ABSTRACT

The present invention relates to a compound of formula (IA) as defined in the specification or a pharmaceutically acceptable sat thereof, in the preparation of a medicament for both the treatment of inflammatory bowel diseases and the prevention of colon cancer.

The present invention relates to novel methods of treating an inflammatory response in intestinal tissues associated with a disease such as inflammatory bowel disease involving either or both the small and large bowel. In particular the present invention relates to a new use of in macrolides for the treatment of the inflammatory bowel disease.

Inflammatory bowel disease (IBD) is the generic term for a disease of unknown cause that produces chronic inflammation or ulceration of the mucosa of the large and small intestine. This inflammatory bowel disease includes such diseases as ulcerative colitis and Crohn's disease.

Ulcerative colitis is a chronic, non-specific IBD which involves a diffuse inflammation in the mucosa of the large intestine causing ulcerative lesions of the colon. Crohn's disease, also known as regional enteritis or colitis granulomatosa, is most frequently located in the small intestine (small bowel), especially in the ileum, but it can affect any part of the bowel, including the rectum. In the latter case the differentiation of Crohn's disease from ulcerative colitis may give rise to diagnostic problems. Generally, the inflammation differs from that of ulcerative colitis by progressing to layers deeper than the mucosa and affecting the epithelium to a lesser degree. Both diseases have become increasingly frequent especially in the developed countries. Therefore, treatment of IBD has become an important problem of modern medicine.

The presently available medical treatments for IBD generally involve drug therapy directed towards the suppression of gastrointestinal inflammation. The most commonly used medicaments to treat IBD are anti-inflammatory drugs such as the salicylates. The salicylate preparations may be effective in treating mild to moderate disease. Examples of salicylates include sulfasalazine, olsalazine, and mesalamine. All of these medications are given orally in high doses for maximal therapeutic benefit. These medicines are not without side effects including heartburn, nausea, vomiting, diarrhea, and headache.

People with more severe IBD can be treated with corticosteroids, such as prednisone and hydrocortisone, which are more potent and faster-acting than salicylates in the treatment of IBD, but are endowed with potentially serious side effects.

In IBD patients that do not respond to salicylates or corticosteroids, medications that suppress the immune system are used. However, immunosuppressants cause increased risk of infection, renal failure, and may increase the need for hospitalization. In severe cases of disease, the patient may need surgery to remove the affected gut. Drugs like antidiarrheals, laxatives, and pain relievers can be also given to help relieve symptoms.

Since all the available medical treatments for IBD are rather unsatisfactory and often ineffective, there is currently a great need for novel drugs capable of treating IBD and preventing its relapse.

We have now found that some macrolides, encompassed by the general formula (I), as reported in the International patent application WO 2004/013153 and endowed with anti-inflammatory activity particularly in skin and lung inflammation, are also surprisingly effective in treating intestinal inflammation.

It is therefore a first object of the present invention the use of a compound of formula (IA)

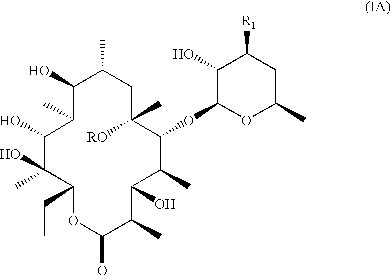

wherein

R is hydrogen or methyl;

R¹ is an N—(C₁-C₄)acyl-N—(C₁-C₃)alkylamino group;

or a pharmaceutically acceptable salts thereof, in the preparation of a medicament for treating IBD.

Examples of pharmaceutically acceptable salts of the compounds of formula (IA) are salts with organic or mineral acids such as hydrogen chloride, hydrogen bromide, hydrogen iodine, nitric acid, sulphuric acid, phosphoric acid, acetic acid, tartaric acid, citric acid, benzoic acid, succinic acid and glutaric acid.

In particular, the compounds of formula (IA) or a pharmaceutically acceptable salt thereof can be used for treating ulcerative colitis or Crohn's disease.

Particularly preferred compound of formula (IA) according to the invention is the compound of formula (ia)

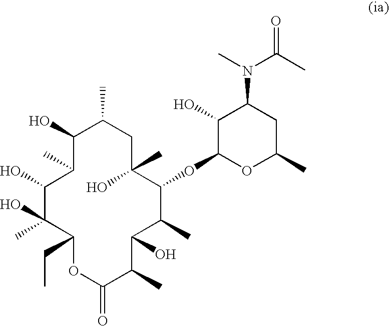

namely, (9S)—O5-[3-oxoethyl)-methylamino-β-D-xylo-3,4,6-trideoxy-hexapyranosyl]-9-hydroxy-9-deoxo-erythronolide-A, or a pharmaceutically acceptable salt thereof.

Unless otherwise specified, the term “treatment” as used herein relates to both treatment in order to cure or alleviate a disease or a condition, and to treatment in order to prevent the development or relapse of a disease or a condition.

The term “patient”, as used herein, relates to any human or non-human mammal in need of treatment according to the invention.

In IBD, the constant process of inflammatory injury and repair of the lining of the colon (colonic mucosa) is believed to make the individual more susceptible to the cancer. Colon cancer doesn't distinguish between active disease and remission. Patients whose disease has been quiet have the same risk as those who have more active disease.

For example, patients with prolonged ulcerative colitis are at increased risk for developing colon cancer. The risk of cancer increases with the duration and the extent of involvement of colonic mucosa. For example, if only the lower colon and rectum are involved, the risk of cancer is no higher than normal. However, if the whole colon is involved, the risk of cancer may be higher.

It is therefore a further object of the present invention the use of a compound of formula (IA) as defined above or a pharmaceutically acceptable salt thereof for the preparation of a medicament for preventing colon cancer.

In a further aspect, the present invention relates to a method for treating IBD, which comprises administering to a patient in need thereof a therapeutically effective amount of a compound of formula (IA) or a pharmaceutically acceptable salt thereof.

In a still further aspect the present invention relates to a method for preventing colon cancer, which comprises administering to a patient in need thereof a therapeutically effective amount of a compound of formula (IA) or a pharmaceutically acceptable salt thereof.

The pharmacological activity of the compound of formula (ia), as a representative compound of the compounds of the present invention, was evaluated in in vivo models of intestinal inflammation, as illustrated in the following EXPERIMENTAL PART.

### BRIEF DESCRIPTION OF THE DRAWINGS

In the Experimental Part below reference is made to the appended drawings on which:

FIG. 1 shows the scheme of drug treatments. Oral drugs were suspended in 1% methocel and given in a volume of 0.5 ml.

FIG. 2 shows the macroscopic damage score criteria. The final score is obtained by the sum of all values.

FIG. 3 shows values of macroscopic damage score indicating the severity of DNBS-induced colitis in rats. Each column indicates the mean value obtained from 6-8 animals±S.E.M. (vertical lines). Significant difference from control values *P<0.05 (one way ANOVA following by Student-Newman-Keuls test).

### EXPERIMENTAL PART

### Methods

### Induction of Colitis

Albino male Sprague-Dawley rats, 200-250 g body weight, were treated with intrarectal DNBS (2,4-dinitrobenzensufonic acid, 30 mg in 0.25 ml of 50% ethanol) under a light anaesthesia with diethyl ether. In control experiments, the animals received 0.25 ml of saline (NaCl 0.9%). Animals were subjected to subsequent experimental procedures 6 days after induction of colitis with DNBS (Blandizzi et al., Altered prejunctional modulation of intestinal cholinergic and noradrenergic pathways by alpha-2-adrenoceptors in the presence of experimental colitis. Br J Pharmacol 139, 309-320, 2003).

### Experimental Design

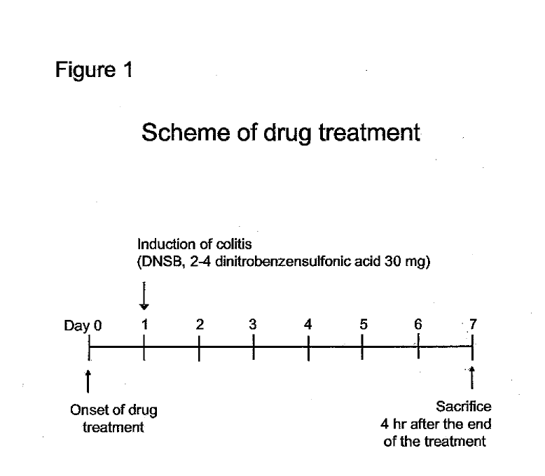

The compound of formula (ia), from now on the macrolide, was suspended in 1% methocel and administered to animals by intragastric gavage in a volume of 0.5 ml at the doses of 100 or 300 μmol/kg/day for 7 consecutive days. Induction of colitis was performed on day 2 as reported above. Animals were subjected to subsequent experimental procedures on day 7, four hours later from administration of the last dose of the macrolide or its vehicle (FIG. 1).

Each experimental series included the following treatment groups:
1) controls (intragastric drug vehicle for 7 days plus intra-colonic saline on day 2);
2) intragastric macrolide for 7 days plus intra-colonic saline on day 2;
3) intragastric drug vehicle for 7 days plus intra-colonic DNBS on day 2;
4) intragastric macrolide for 7 days plus intra-colonic DNBS on day 2.

Each data point represents the mean value of results obtained from at least 6-8 animals. The effect of the macrolide was compared with that of dexamethasone (1 mg/kg/day, by oral route), known to exert a good anti-inflammatory action in the rat model of TNBS-induced colitis (Bobin-Dubigeon et al., Effects of tumor necrosis factor-α synthesis inhibitors on rat trinitrobenzene sulfonic acid-induced chronic colitis. Eur J Pharmacol 431, 103-110, 2001). Dexamethasone was administered once daily following the same time schedule adopted for the macrolide.

### Macroscopic and Histological Assessment of Intestinal Inflammation

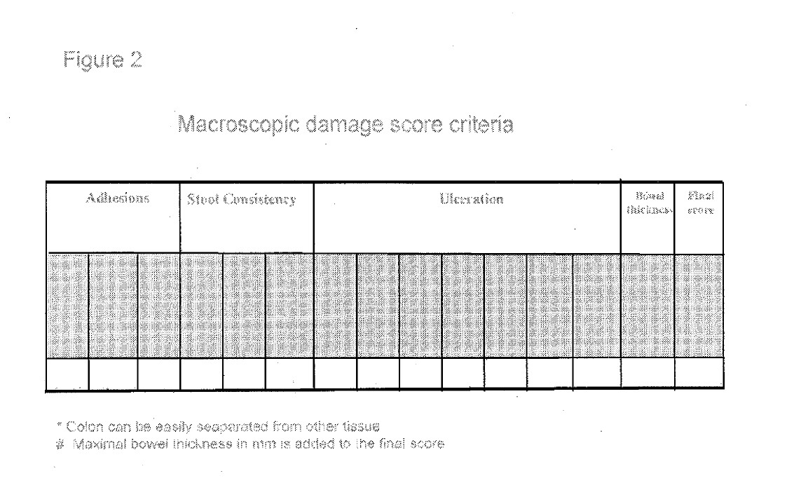

At the end of treatments, animals were sacrificed and the severity of colonic inflammation was macroscopically evaluated in accordance with the criteria previously reported by Wallace and Keenan (Wallace J L and Keenan C M, An orally active inhibitor of leukotriene synthesis accelerates healing in a rat model of colitis. Am J Physiol, 258, G527-G534, 1990) with minor modifications (Blandizzi, et al., 2003). Briefly, the macroscopic evaluation is based on the following criteria: presence of adhesions between the colon and other intra-abdominal organs; consistency of colonic faecal material (as an indirect marker of diarrhea); thickening of the colonic wall; presence and extension of hyperemia and macroscopic mucosal damage (assessed with the aid of a ruler) (FIG. 2). All parameters of macroscopic damage were recorded and scored for each rat by two independent observers blinded to the treatment.

### Results

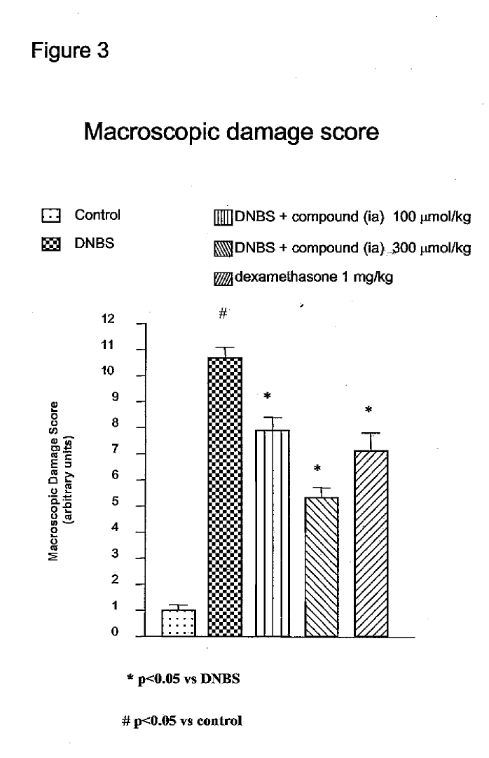

The obtained result are illustrated in FIG. 3. Control rats exhibited a negligible macroscopic damage score, accounting for 1.0±0.2. In DNBS-treated animals, the extent of inflammatory lesions was significantly higher than that observed in control rats (10.7±0.4), indicating the occurrence of colitis. The macrolide exerted significant anti-inflammatory effects both at 100 and 300 μmol/kg (−24% and −51%, respectively). Dexamethasone induced anti-inflammatory effect similar to that observed.with the macrolide 100 μmol/kg.

### Conclusions

Data obtained from the present study indicate a good pattern of anti-inflammatory activity of the macrolide against experimental colitis. In this setting, the efficacy of the macrolide seems to be comparable with that of dexamethasone, a well known anti-inflammatory drag.

It is thus clear from the experimental evidence reported above that a compound of formula (IA), especially the compound of formula (ia) or a pharmaceutically acceptable salt thereof, may be effectively used in the treatment of IBD and in the prevention of colon cancer.

The therapeutical effective amounts of a compound of formula (IA) or a pharmaceutically acceptable salt thereof will depend on the age and the general physiological state of the patient, the route of administration and the pharmaceutical formulation used; the therapeutic doses will generally be between about 10 and 2000 mg/day and preferably between about 30 and 1500 mg/day.

The compounds of the present invention for use in the treatment and/or prophylaxis of the above mentioned diseases will preferably be used in a pharmaceutical form that is suitable for oral rectal, sublingual, parenteral, topical transdermal and inhalational administration. It is therefore a further object of the present invention to provide pharmaceutical formulations containing a therapeutical effective amount of a compound of formula (IA) or a pharmaceutically acceptable salt thereof together with a pharmaceutical acceptable vehicle.

The pharmaceutical formulations of the present invention may be liquid, suitable for oral and/or parenteral administration, for instance drops, syrups, solutions, injectable solutions ready to use or prepared via dilution of a lyophilizate, but preferably solid, for instance tablets, capsules, granules, powders, pellets, pessaries, suppositories, creams, pomades, gels or ointments; or alternatively solutions, suspensions, emulsions or other forms suitable for inhalation and transdermal administration.

Depending on the type of formulation, these formulations will contain, besides a therapeutical effective amount of a compound of formula (IA) or a pharmaceutically acceptable salt thereof, solid or liquid excipients or diluents for pharmaceutical use and optionally other additives normally used in the preparation of pharmaceutical formulations, for instance thickeners, aggregating agents, lubricants, disintegrants, flavorings and colorings.

The pharmaceutical formulations according to the invention, may be produced according to conventional techniques. As an example, hard gelatine capsules for oral administration comprising from 100 mg to 300 mg of the macrolide and a pharmaceutically acceptable vehicle may be prepared to be administered to a patient in need thereof.

1. A method of treating inflammatory bowel disease, comprising administering an effective amount of a compound of formula (IA) 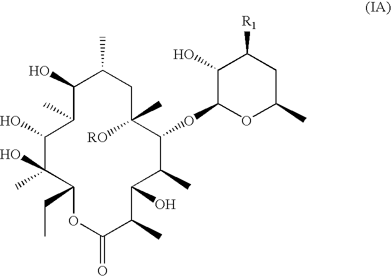
wherein
R is hydrogen or methyl;
R1 is an N—(C1-C4)acyl-N—(C1-C3)alkylamino group;
or a pharmaceutically acceptable salts thereof, to a patient in need thereof.

2. The method according to claim 1, wherein the compound of formula (IA) is (9S)—O5-[3-(1-oxoethyl)-methylamino-β-D-xylo-3,4,6-trideoxy-hexapyranosyl]-9-hydroxy-9-deoxo-erythronolide-A, or a pharmaceutically acceptable salt thereof.

3. The method according to claim 1, wherein the inflammatory bowel disease is ulcerative colitis.

4. The method according to claim 1, wherein the inflammatory bowel disease is Crohn's disease.

5. A method, for the prevention of colon cancer, comprising administering an effective amount of a compound of formula (Ia) 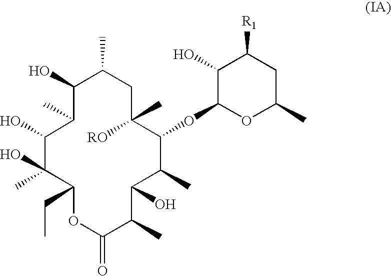
wherein
R is hydrogen or methyl;
R1 is an N—(C1-C4)acyl-N—(C1-C3)alkylamino group;
or a pharmaceutically acceptable salts thereof, to a patient in need thereof.

6. The method according to claim 5, wherein the compound of formula (IA) is (9S)—O5-[3-(1-oxoethyl)-methylamino-β-D-xylo-3,4,6-trideoxy-hexapyranosyl]-9-hydroxy-9-deoxo-erythronolide-A, or a pharmaceutically acceptable salt thereof.

7.

8. The method of claim 1, wherein the amount of the compound of formula (IA) administered is between about 10 to 2,000 mg/day.

9. The method of claim 1, wherein the amount of the compound of formula (IA) administered is between about 30 to 1,500 mg/day.
